(19) United States
(12) Patent Application Publication
(10) Pub. No.: US 2003/0170731 A1
(43) Pub. Date: Sep. 11, 2003

(54) Diagnosis and treatment of non-ulcer dyspepsia based on hypothalamic-pituitary-adrenal axis abnormallity

(76) Inventors: Timothy Garard Dinan, Cobh (IE); Paul William Napoleon Keeling, Donnybrook (IE)

Correspondence Address: BIRCH STEWART KOLASCH & BIRCH, PO BOX 747, FALLS CHURCH, VA, 22040-0747, US

(21) Appl. No.: 10/343,433

(22) PCT Filed: Jul. 30, 2001
(86) PCT No.: PCT/IE01/00098

(30) Foreign Application Priority Data
Aug. 2, 2000 (IE) 20000616

(51) Int. Cl. G01N 33/53; A61K 31/53; A61K 31/519

(52) U.S. Cl. 435/7.1; 514/262.1; 514/246

(57) ABSTRACT

An in vitromethod and a kit for the diagnosis of non-ulcer dyspepsia (NUD) in a subject suspected of having NUD, comprises identifying in the subject a dysfunction of corticotropin releasing hormone (CRH) receptors characterised by the subject's response to CRH-induced adrenocorticotropic hormone (ACTH) production by estimating the level of ACTH in a sample of blood or a blood fraction obtained from said subject relative to a control. The method can be used to diagnose NUD in patients who are infected with Helicobacter pylori and those who are free of such infection.

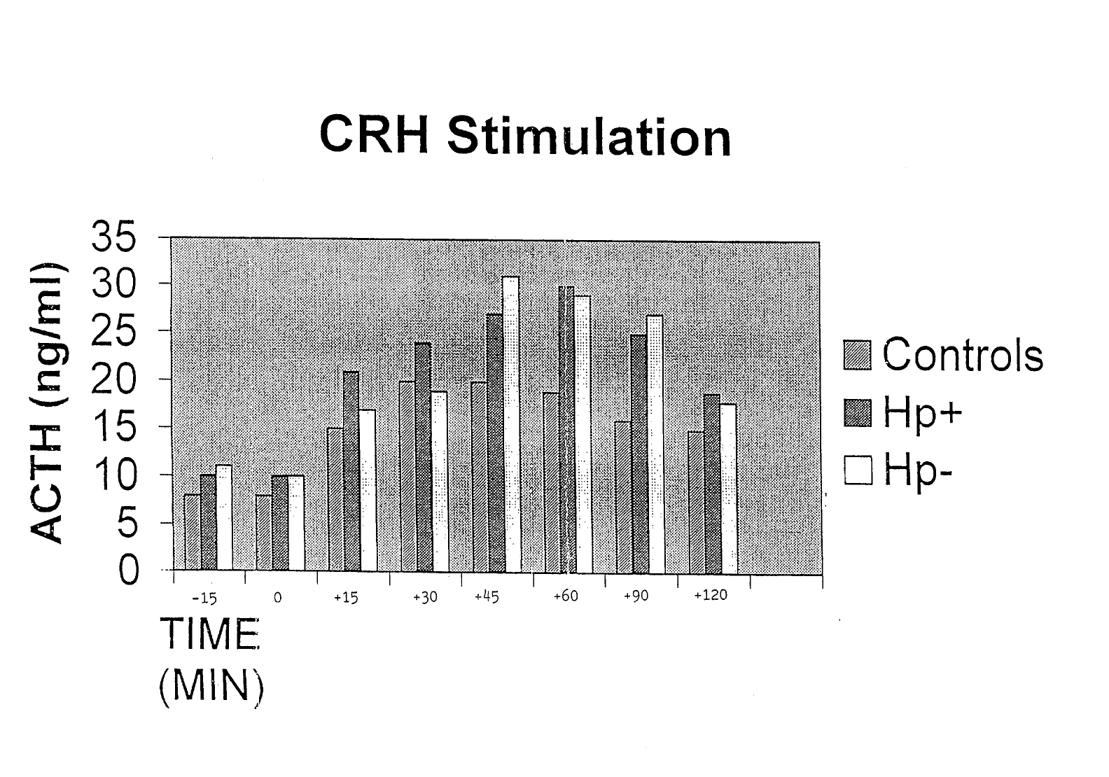

### TECHNICAL FIELD

This invention relates to the diagnosis and therapy of non-ulcer dyspepsia (NUD), for patients who have Helicobacter pylori infection and those who do not.

### BACKGROUND ART

Symptoms of post prandial fullness or bloating, early satiety, excessive flatulence, upper abdominal pain and nausea are commonplace both in primary care and in gastroenterology clinics (Fisher, R. S. and Parkman, H. P. New England J Med (1998), 339, 1376-1381). These symptoms are sometimes attributable to H. pylori infection, however in many cases no organic disease is found. The substantial group of patients who have persistent and occasionally disabling gastric symptoms without organic disease are classified as having functional dyspepsia or NUD (Talley, N. J. et al, Gastroenterology (1992) 102, 1259-68.

Non-ulcer dyspepsia is a heterogeneous condition and has been classified according to symptom clusters as dysmotility-like dyspepsia, gastrooesophageal reflux-like dyspepsia, aerophagia, and essential dyspepsia (Talley, N. J. and Philips, S. F., Ann. Inter. Med. (1988) 108, 865-9). NUD is the most common reason for referral to gastroenterology clinics. To date systematic efforts to determine its aetiology have failed. No consistent biochemical or physiological abnormalities have been demonstrated and many gastroenterologists describe this condition as a functional disorder without an organic basis.

The role of H. Pylori in the genesis of peptic ulceration is now well established (Marshall, B. J. et al., Lancet (1988) 2, 1437-42). Some patients with a diagnosis of NUD have evidence of H. pylori infection. However, many of these patients continue to complain of symptoms even when H. pylori has been eradicated (Fisher, R. S. and Parkman, H. P., (1998) supra). In these patients the persistence of symptoms is not related to chronic infection (Tally N. J. et al. BMJ (1999) 318, 833-837)

That stress plays an influential role in the genesis of the disorder has long been suspected and hence the name functional dyspepsia.

The core stress axis in man is the hypothalamic-pituitary-adrenal axis (HPA). This axis is essential in enabling the organism to withstand stress of either a physical or a psychological nature. In response to stress the axis is activated and without such activation the organism cannot survive. Patients with Addison's disease (characterised by adrenal cortical insufficiency) require increased glucocorticoid supplementation during situations of stress.

Corticotropin releasing hormone (CRH) is produced in the paraventricular nucleus in the hypothalamus and is the dominant peptide regulator of the axis (Scott, L. V. and Dinan, T. G., Life Sciences (1998) 62, 1985-98). In situations of chronic stress many neurons in the paraventricular nucleus co-express vasopressin. In these circumstances it is thought that vasopressin plays the dominant role in controlling the axis. Both peptides act synergistically in stimulating ACTH release from the anterior pituitary. This in turn stimulates the production and release of cortisol from the adrenal cortex. A series of negative feedback loops regulate the axis. These feedback loops are defined according to the timeframe as either immediate, intermediate or delayed.

A large number of stress-related conditions are associated with abnormalities in the HPA. Major depressive illness is the best characterised of these conditions. Patients with major depression hypersecrete cortisol and frequently fail to suppress cortisol in response to dexamethasone challenge (Dinan T. G., Brit. Journal of Psychiatry (1994) 21, 813-29). When patients with major depression are administered CRH intravenously their output of ACTH is reported to be blunted.

Studies in animals suggest that chronic infections increase vasopressin production and result in ACTH blunting in response to CRH infusion. Such findings demonstrate that HPA disturbances can be brought about by either psychological or physical stressors.

So far the only bowel disorder investigated inrelation to HPA activity is colonic irritable bowel syndrome a disorder characterised by colicky abdominal pain and alteration of bowel habit in the absence of obvious pathology. Irritable Bowel Syndrome (IBS) and NUD are traditionally regarded as separate clinical entities. Patients can have both IBS and NUD. Fukudo, S. et al., (Gut (1998) 42, 845-9) reported that such patients show an enhanced or exaggerated ACTH response to CRH infusion.

EP-A 0 720 850 describes and claims an in vitro method for the diagnosis of NUD which involves identifying in a subject a dysfunction of central 5-HT 1A receptors which is characterised by the subject's response to azaspirodecanedione induced prolactin production by estimating the level of prolactin in a sample of blood fraction obtained from said subject, with the proviso that when the subject is a menstruating female, the diagnosis is carried out in the follicular phase of menstruation.

### DISCLOSURE OF INVENTION

The invention provides an in vitro method for the diagnosis of non-ulcer dyspepsia (NUD) in a subject suspected of having NUD, which method comprises identifying in said subject a dysfunction of corticotropin releasing hormone (CRH) receptors characterised by the subject's response to CRH-induced adrenocorticotropic hormone (ACTH) production by estimating an increased level of ACTH in a sample of blood or a blood fraction obtained from said subject relative to a control.

The method according to the invention can be used to diagnose NUD in patients who are infected with Helicobacter pylori and those who are free of such infection.

Preferably, the increased level of ACTH in the sample of blood or blood fraction results from the subject having been administered CRH as a single dose in an amount of the order of 100 μg prior to said in vitro identification step.

Further, preferably, the CRH is administered intravenously. When CRH is given intravenously to patients, it is either given as hCRH or oCRH, hCRH being the human variant of the molecule and oCRH the ovine variant.

Preferably, the CRH is oCRH.

The ovine variant is preferred because it gives more sustained endocrine responses in man.

The blood fraction is preferably plasma.

The ACTH response is suitably measured by immunoassay, more particularly immunoradiometric assay.

The labelled agents for use in such assays can be prepared in conventional manner or can be purchased from appropriate suppliers.

The invention also provides a kit for carrying out the method hereinbefore described, which includes an amount of CRH of the order of 100 μg for administering to one or more subject(s) being investigated for NUD and sufficient to elicit an ACTH response characteristic of CRH receptor dysfunction.

The kit will preferably include the necessary components/ingredients for carrying out the ACTH estimation.

Accordingly, the CRH/ACTH test described herein is a suitable diagnostic test for NUD either with Helicobacter pylori infection or not. It will be appreciated, therefore, that CRH antagonists would be effective in the management of NUD.

In a further aspect of the invention there is provided use of a CRF(1) receptor antagonist in the manufacture of a medicament for use in the treatment of non-ulcer dyspepsia.

Preferably, the CRF(1) receptor antagonist is selected from a 3-phenylpyrazolo[1,5-a]pyrimidine and a pyrazolo[1,5a-]-1,3,5-triazine.

Further, preferably, the CRF(1) receptor antagonist is 4-(1,3-dimethoxyprop-2-ylamino)-2,7-dimethyl-8-(2,4-dichlorophenyl)pyrazolo[1,5-a]-1,3,5-triazine.

The terms CRH and CRF (corticotropin releasing factor) are used interchangeably. However, CRF should only be termed CRH when it is used as a hormone. In certain circumstances CRF has a role outside of the endocrine system.

The results described herein indicate that a peripheral response to NUD purely in the form of H. pylori eradication is unlikely to produce long-term symptom relief. The results described herein places the emphasis on the brain and HPA in mediating the symptoms of NUD.

Although not wishing to be bound by any theoretical explanation of the invention, it is proposed that CRH receptor supersensivity in the hypothalamic-pituitary axis and perhaps in the brain is central to the pathophysiology of NUD. It is proposed that this alternation in responsivity is induced in the case of psychological stress via neurotransmitter inputs to the hypothalamis and in the case of H. pylori by cytokine production and stimulation of paraventricular neurons. The cytokines IL-1 and IL-6 are the most potent stimuli of the HPA.

### BRIEF DESCRIPTION OF THE DRAWING

The accompanying FIGURE is a graph of plasma ACTH (ng/ml) concentration following CRH stimulation as described in the Example versus time (min.) for NUD patients with H. pylori (Hp+) and NUD patients without H. pylori (Hp−) relative to healthy volunteers (control).

The invention will be further illustrated by the following Example.

### MODE FOR CARRYING OUT THE INVENTION

### Example

A total of ten patients with NUD were recruited from the Gastroenterology Clinic at St. James's Hospital, Dublin. Patients consisted of six men and four woman between the ages of twenty eight and forty five. They were matched for age and sex with ten healthy volunteers. To be included in the study patients had at least four of the following symptoms for at least three months: early satiety, upper abdominal pain, post prandial bloating, excessive flatulence, borborygmus, and nausea and/or vomiting. They all had normal results on physical examination. Endoscopic examination, twenty-four hour ambulatory pH monitoring, abdominal ultrasound were all negative. Five patients with H. pylori infestation were tested both before and after H. pylori eradication. The other five patients had no evidence of H. pylori infection. None of the patients had evidence of a formal psychiatric illness. Also none of the patients fulfilled the criteria for IBS.

Patients who were H. pylori positive were re-tested six months after commencing eradication therapy. In all cases endocrine tests were commenced at 1400 h. The test procedure was as follows. An 18 g cannula was inserted in a forearm vein at 1330 h and the subjects relaxed for 30 min before baseline blood for ACTH estimation was drawn. At this point ovine CRH 100 μg was administered intravenously and further blood for ACTH measurement was taken at 15, 30, 45, 60, 90 and 120 min.

Plasma ACTH was measured using a two site unextracted immunoradiometric assay with a commercially available kit supplied by the Nichols Institute (San Juan Capistrano, Calif.). The sensitivity of the assay is 5 ng per 1. Intra-assay and inter-assay coefficient of variation were 3% and 6% respectively.

As can be seen from the accompanying Figure patients with NUD show vastly enhanced ACTH responses relative to the healthy volunteers. This enhancement is seen both in patients with H. pylori infection and those without. Furthermore, following eradication therapy it is clear that the CRH/ACTH enhancement persists. This finding may help explain why so many patients following eradication therapy continue to complain of symptoms.

1. An in vitro method for the diagnosis of non-ulcer dyspepsia (NUD) in a subject suspected of having NUD, which method comprises identifying in said subject a dysfunction of corticotropin releasing hormone (CRH) receptors characterised by the subject's response to CRH-induced adrenocorticotropic hormone (ACTH) production by estimating an increased level of ACTH in a sample of blood or a blood fraction obtained from said subject relative to a control.

2. A method according to claim 1, wherein the increased level of ACTH in the sample of blood or blood fraction results from the subject having been administered CRH as a single dose in an amount of the order of 100 μg prior to said in vitro identification step.

3. A method according to claim 1 or 2, wherein the blood fraction is plasma.

4. A method according to claim 1, substantially as hereinbefore described and exemplified.

5. A kit for carrying out a method according to any one of claims 1-4, which includes an amount of CRH of the order of 100 μg for administering to one or more subject(s) being investigated for NUD and sufficient to elicit an ACTH response characteristic of CRH receptor dysfunction.

6. Use of a CRF(1) receptor antagonist in the manufacture of a medicament for use in the treatment of non-ulcer dyspepsia.

7. Use according to claim 6, wherein the CRF(1) receptor antagonist is selected from a 3-phenylpyrazolo[1,5-a]pyrimidine and a pyrazolo[1,5-a]-1,3,5-triazine.

8. Use according to claim 6 or 7, wherein the CRF(1) receptor antagonist is 4-(1,3-dimethoxyprop-2-ylamino)-2,7-dimethyl-8-(2,4-dichlorophenyl)pyrazolo[1,5-a]-1,3,5-triazine.
